(19) United States
(12) Patent Application Publication
(10) Pub. No.: US 2022/0057373 A1
(43) Pub. Date: Feb. 24, 2022

(54) SYSTEM AND METHOD FOR MONITORING DIESEL VEHICLE EMISSIONS BASED ON BIG DATA OF REMOTE SENSING

(71) Applicant: Beijing Institute of Technology, Beijing (CN)

(72) Inventors: Lijun HAO, Beijing (CN); Zihao GE, Beijing (CN); Hang YIN, Beijing (CN); Xiaohu WANG, Beijing (CN); Junfang WANG, Beijing (CN); Jin LIU, Beijing (CN); Miao TIAN, Beijing (CN); Mingliang FU, Beijing (CN)

(73) Assignee: Beijing Institute of Technology, Beijing (CN)

(21) Appl. No.: 17/166,531

(22) Filed: Feb. 3, 2021

(30) Foreign Application Priority Data
Aug. 18, 2020 (CN) 202010832913.5

(51) Int. Cl. G01N 33/00; G01N 21/33

(52) U.S. Cl. CPC G01N 33/0037; G01N 2021/1793; G01N 21/33

(57) ABSTRACT

The present disclosure provides a system and method for monitoring diesel vehicle emissions based on big data of remote sensing. The monitoring system includes a vehicle remote sensing data monitoring platform, a host computer, an emission remote sensing instrument, a vehicle driving state monitor, an information display screen and a license plate camera. The emission remote sensing instrument is used to acquire information of a pollutant in an exhaust plume. The vehicle driving state monitor is used to acquire a vehicle speed and acceleration. The license plate camera is used to capture license plate information. The host computer is used to process and calculate vehicle cycle and emission information. The vehicle remote sensing data monitoring platform is used to determine a high-emission vehicle, and pre-store information of all diesel vehicles, different driving cycle bins of each type of diesel vehicles and high-emission thresholds set for different bins.

### PRIORITY

This application claims priority of Chinese patent application number 202010832913.5 filed on Aug. 18, 2020 the contents of which is fully incorporated herein by reference.

### TECHNICAL FIELD

The present disclosure relates to the technical field of motor vehicle exhaust detection, in particular, to a system and method for monitoring diesel vehicle emissions based on big data of remote sensing.

### BACKGROUND

Diesel engines of vehicles emit less hydrocarbon (HC) and carbon monoxide (CO), and the main emission control lies in nitrogen oxides (NOₓ) and particulate matter (PM). NOₓ is toxic to the human respiratory system. NOₓ and HC are prone to generate photochemical smog under the action of sunlight, which increases the concentration of ozone in the atmosphere, and causes great harm to the surrounding environment. PM affects the atmospheric visibility, and has a strong adsorption, adsorbing substances with strong toxic effects. After PM is inhaled into the human body, it will cause physical discomfort and easily induce respiratory diseases. In severe cases, it will enter the human blood and cause myocardial infarction and cardiovascular diseases, etc. At present, the NOₓ and PM emissions of diesel vehicles that are less than 10% of the total number of vehicles have reached 70% and 90% of the total NOₓ and PM emissions of vehicles respectively, which have become the top priority of prevention and control of motor vehicle pollution.

At present, the emission test of diesel vehicles in use in China mainly adopts the lug-down method to test NOₓ and exhaust smoke. When the lug-down mode is not met, especially during road inspections, the traditional free acceleration smoke test method can only be used. Due to the limitation of the test equipment, the diesel vehicle is stationary to test the free acceleration smoke. Although the operation is simple, there will be large errors with the exhaust smoke in the actual driving cycle. The period of regular environmental inspections for in-use vehicles generally ranges from 6 months to 2 years, which is too long to ensure that the vehicles meet emission standards during the inspection period. Therefore, there is an urgent need for a convenient, rapid and effective method for detecting emissions of in-use vehicles.

At present, the remote sensing detection equipment is technically feasible in the emission detection of gasoline vehicles but has large errors in the NOₓ emission detection of diesel vehicles. The remote sensing detection system has a high misjudgment rate in screening high-emission diesel vehicles, which means that the current remote sensing detection system is not yet mature for the NOₓ emission detection of diesel vehicles.

The NOₓ emission and exhaust smoke of diesel vehicles vary with the driving cycle. Meanwhile, different types of diesel engines have different transmissions and other configurations, and their working conditions and loads vary greatly, resulting in significant differences in the emission characteristics of NOₓ and smoke. At present, the diesel vehicle remote sensing detection standard only specifies a single limit of NOₓ emission concentration or exhaust smoke opacity for the screening of high-emission diesel vehicles, which is equivalent to classifying all driving cycles of all types of vehicles into one bin. This treatment method is not conducive to the scientific and refined management of diesel vehicles. It is easy to cause high-emission vehicles to escape inspection and punishment when they run with low emissions at low load or low speed conditions, and to misjudge some low-emission vehicles as high-emission vehicles due to their high emissions under high loads. This is also one of the main reasons for the misjudgment of the current remote sensing detection for diesel vehicle emissions.

Therefore, it is an urgent problem to be solved by those skilled in the art to provide a remote sensing monitoring system and method for diesel vehicle emissions.

### SUMMARY

In view of this, the present disclosure provides a system and method for monitoring diesel vehicle emissions based on big data of remote sensing. The present disclosure realizes the real-time measurement of diesel vehicle emissions and is suitable for exhaust emission detection of various motor vehicles powered by diesel engines. The present disclosure features convenient operation, rapidness, high efficiency and high accuracy.

To achieve the above objective, the present disclosure adopts the following technical solutions:

An system for monitoring diesel vehicle emissions based on big data of remote sensing, including: a vehicle remote sensing data monitoring platform, a host computer, an emission remote sensing instrument, a vehicle driving state monitor, an information display screen and a license plate camera, where the emission remote sensing instrument, the vehicle driving state monitor, the information display screen and the license plate camera are all connected to the host computer; the host computer is connected to the vehicle remote sensing data monitoring platform via the Internet;

the emission remote sensing instrument is used to acquire information of a pollutant in an exhaust plume of a vehicle under inspection;

the vehicle driving state monitor is used to acquire a speed and an acceleration of the vehicle under inspection;

the information display screen is used to display relevant information of the vehicle under inspection;

the license plate camera is used to capture license plate information of the vehicle under inspection;

the host computer is used to acquire information, process data and calculate a concentration of the pollutant in the exhaust plume;

the vehicle remote sensing data monitoring platform is used to receive vehicle cycle information and emission data transmitted by the host computer, determine a high-emission vehicle and store relevant information to a database, where the database pre-stores information of all diesel vehicles, different driving cycle bins of each type of diesel vehicles and high-emission thresholds set for different bins.

A monitoring method using a system for monitoring diesel vehicle emissions based on big data of remote sensing, including:

step 1: detecting a speed and an acceleration of a vehicle under inspection through a vehicle driving state monitor, detecting an intensity of detection light passing through an exhaust plume by an emission remote sensing instrument, capturing license plate information of the vehicle under inspection by a license plate camera, and sending the above information to a host computer;

step 2: calculating concentrations of CO, HC, NO and CO₂ in the exhaust plume by the host computer according to the detection light intensity detected by the emission remote sensing instrument; determining an air-fuel ratio (AFR) of a diesel engine of the vehicle under inspection according to the speed, acceleration and type of the vehicle under inspection, determining a NO emission level, and calculating an exhaust smoke opacity of the diesel vehicle based on the intensity of the detection light passing through the exhaust plume; and

step 3: receiving, by a vehicle remote sensing data monitoring platform, the type, driving cycle and emission information of the vehicle under inspection sent by the host computer; determining the type of the vehicle under inspection; allocating the vehicle's driving cycle parameters, NO emission and exhaust smoke opacity data to a bin corresponding to the vehicle type according to the vehicle speed and acceleration; performing statistical analysis on the emission data, and screening a high-emission vehicle.

According to the above technical solutions, the present disclosure provides a system and method for monitoring diesel vehicle emissions based on big data of remote sensing. Compared with the prior art, the present disclosure realizes the real-time measurement of diesel vehicle emissions and is suitable for exhaust emission detection of various motor vehicles powered by diesel engines. In addition, the present disclosure features convenient operation, rapidness, high efficiency and high accuracy.

### BRIEF DESCRIPTION OF DRAWINGS

To describe the technical solutions in the embodiments of the present disclosure or in the prior art more clearly, the following briefly describes the accompanying drawings required for describing the embodiments or the prior art. Apparently, the accompanying drawings in the following description show merely some embodiments of the present disclosure, and a person of ordinary skill in the art may still derive other drawings from these accompanying drawings without creative efforts.

FIG. 1 is a schematic diagram of a system for monitoring diesel vehicle emissions based on big data of remote sensing according to the present disclosure.

FIG. 2 shows the classification of diesel vehicles corresponding to different driving cycle bins according to an embodiment of the present disclosure.

FIG. 3 is a schematic diagram of a cumulative distribution probability of an emission data variable x according to an embodiment of the present disclosure.

FIG. 4 is a schematic diagram of a method for determining an emission determination threshold for high-emission vehicles with a proportion of y % according to an embodiment of the present disclosure.

FIG. 5 shows a map of driving cycle bins and air-fuel ratios (AFRs) of a light-duty diesel passenger vehicle according to an embodiment of the present disclosure.

FIG. 6 a-c shows a cumulative distribution probability of a measured value of an exhaust smoke opacity of a light-duty diesel passenger vehicle under free acceleration according to an embodiment of the present disclosure, where FIG. 6(a) shows a cumulative distribution probability of a measured value of an exhaust smoke opacity of a light-duty diesel passenger vehicle under free acceleration, FIG. 6(b) shows a cumulative distribution probability of a measured value of an exhaust smoke opacity of a light-duty diesel passenger vehicle under free acceleration with 5% high-emission light-duty diesel passenger vehicles screened, and FIG. 6(c) shows a cumulative distribution probability of a measured value of an exhaust smoke opacity of a light-duty diesel passenger vehicle under free acceleration with 15% high-emission light-duty diesel passenger vehicles screened.

### DETAILED DESCRIPTION

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts should fall within the protection scope of the present disclosure.

### Embodiment 1

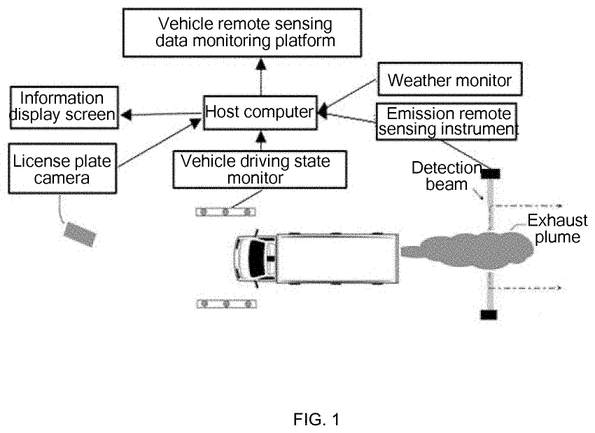

As shown in FIG. 1, this embodiment of the present disclosure provides a system for monitoring diesel vehicle emissions based on big data of remote sensing. The monitoring system includes: an emission remote sensing instrument, a host computer, an information display screen, a vehicle driving state monitor, a vehicle remote sensing data monitoring platform and a license plate camera.

The emission remote sensing instrument, the information display screen, the vehicle driving state monitor and the license plate camera are all connected to the host computer; the host computer is connected to the vehicle remote sensing data monitoring platform via the Internet.

The vehicle driving state monitor includes an optical or radar measuring instrument for vehicle speed and acceleration. The vehicle driving state monitor is disposed beside a road in a vehicle detection area and is able to accurately measure the speed and acceleration of a vehicle under inspection when the vehicle passes by.

The emission remote sensing instrument adopts a vertical or horizontal optical path and is disposed in a passing area of a vehicle. In order to ensure the accuracy of emission remote sensing of the diesel vehicle, the road surface where the emission remote sensing instrument is disposed must be level or slightly uphill so that the diesel vehicle is running at a constant speed or slightly accelerating to obtain the best emissions detection results. Therefore, it is more appropriate to set up remote sensing monitoring points of exhaust emissions at the entrance of the bridge approach, the passage to leave the highway toll station and the slightly uphill road. The emission remote sensing instrument includes a detection light emitting device, a detection light receiving device and a detection light reflecting device. The detection light emitting device is used to emit detection light, and the detection light receiving device is used to receive the detection light passing through an exhaust plume.

The information display screen is a high-brightness lattice screen, which is able to display relevant information of the vehicle under inspection in real time, including information such as license plate number, vehicle speed and exhaust pollutant concentration.

The license plate camera is a high-speed camera, which is able to accurately capture license plate information. The license plate camera may also be other image recognition equipment that can obtain license plate information, which is not limited in the present disclosure.

The host computer may be an industrial control computer, which is responsible for: acquiring and processing all the above input and output signals and system calibration, completing exhaust emission calculation, sending data to the vehicle remote sensing data monitoring platform via the Internet, and communicating with the vehicle remote sensing data monitoring platform.

The vehicle remote sensing data monitoring platform receives type, driving cycle and emission information of the vehicle under inspection sent by the host computer, determines the type of the vehicle under inspection, allocates the vehicle's driving cycle parameters, NO emission level and exhaust smoke opacity data to a bin corresponding to the vehicle type according to the vehicle speed and acceleration, performs statistical analysis on the emission data, and determines a high-emission vehicle. The vehicle remote sensing data monitoring platform has a database, which pre-stores information of all diesel vehicles, different driving cycle bins of each type of diesel vehicles and high-emission thresholds set for different bins.

The system further includes a weather monitor, which is connected to the host computer and is used to acquire environmental information of an area where the vehicle under inspection passes. In case of severe weather such as storms, the system stops detection to prevent inaccurate detection data due to natural factors. The weather monitor is a miniature weather station, which is also disposed in the passing area of the vehicle and is able to accurately measure environmental parameters, such as wind speed, wind direction, temperature, humidity and other information.

In this embodiment, the vehicle driving state monitor measures a speed and an acceleration of the diesel vehicle. The host computer uses the speed and acceleration of the diesel vehicle as parameters and calculates an air-fuel ratio (AFR) of the diesel vehicle in the current driving cycle by interpolating an AFR map of the diesel vehicle. Meanwhile, the host computer calculates emission concentration ratios of CO, HC and NO to CO₂ in the exhaust plume based on a light intensity detected by the emission remote sensing instrument, and calculates emission concentrations of CO, HC, NO and CO₂ in the diesel vehicle exhaust according to the AFR of the diesel vehicle in the current driving cycle, so as to realize the real-time measurement of the gaseous emission concentrations in the diesel vehicle exhaust.

The remote sensing test of the exhaust smoke opacity of the diesel vehicle uses multiple pairs of optical paths provided by photoelectric sensors, where one side of the optical path emits a detection beam, and the other side thereof receives the beam. Alternatively, the detection beam emitting device and the receiving device are placed on the same side, and a reflecting device is placed on the other side to reflect the detection beam emitted by the emitting device back to the receiving device. A light source works in a modulation mode, which effectively avoids the influence of ambient light on the measurement. The beam range covers the height of the tailpipe of most motor vehicles, and can obtain a measurement result of a vertical section of the exhaust smoke of the diesel vehicle. There are 100 levels of light transmittance, and the remote sensing test result of the exhaust smoke is represented by opacity (%).

This system is suitable for the exhaust emission detection of various motor vehicles equipped with diesel engines, and features convenient, rapid and efficient operation.

The embodiment of the present disclosure provides a method for monitoring diesel vehicle emissions based on big data of remote sensing.

The driving cycle of diesel vehicles is divided into multiple small intervals, called bins, by using a speed and an acceleration of the diesel vehicles as parameters. In each bin, the NOₓ emission and smoke opacity of the diesel vehicles are statistically analyzed, and a screening threshold is determined to realize the screening of high-emission vehicles. The remote sensing test database is dynamically updated in real time, which realizes the evaluation of the average emission level of vehicles and the dynamic update of high-emission thresholds for different driving cycle bins of each type of vehicles.

The emission remote sensing instrument operates and records automatically by the host computer, and transmits the vehicle image, license plate information and emission data to the vehicle remote sensing data monitoring platform. The vehicle remote sensing data monitoring platform retrieves the production year, manufacturer, model and owner of the vehicle in the database through the vehicle image and license plate information. Based on the big data method, the present disclosure realizes the screening of high-emission diesel vehicles, the evaluation of the average emission level of the vehicle and the ranking and evaluation of the number of excess emission records and emission levels of all types of vehicles on the market. In this way, spot checks and supervision can focus on a vehicle type with a higher proportion of excess emission records.

### Embodiment 2

The present disclosure provides a method for monitoring diesel vehicle emissions based on big data of remote sensing, which uses the system for monitoring diesel vehicle emissions based on big data of remote sensing provided by Embodiment 1. According to the different emission characteristics of diesel vehicles under different driving cycles, this monitoring method achieves the scientific and refined management of diesel vehicle emissions by classifying the diesel vehicles into different types corresponding to different driving cycle bins. Specifically, this method includes steps S101 to S111:

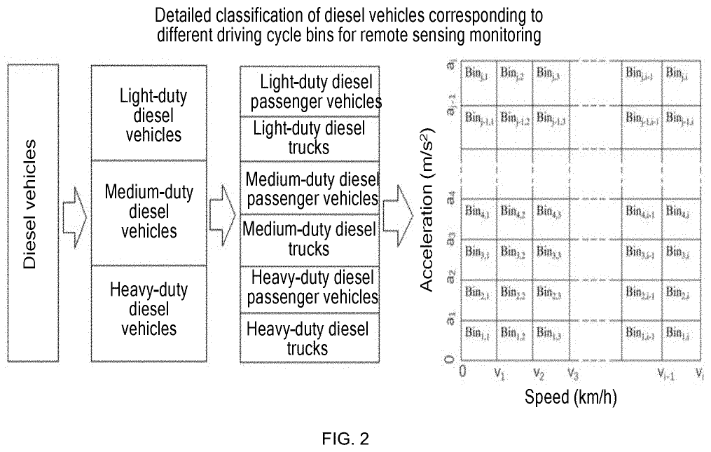

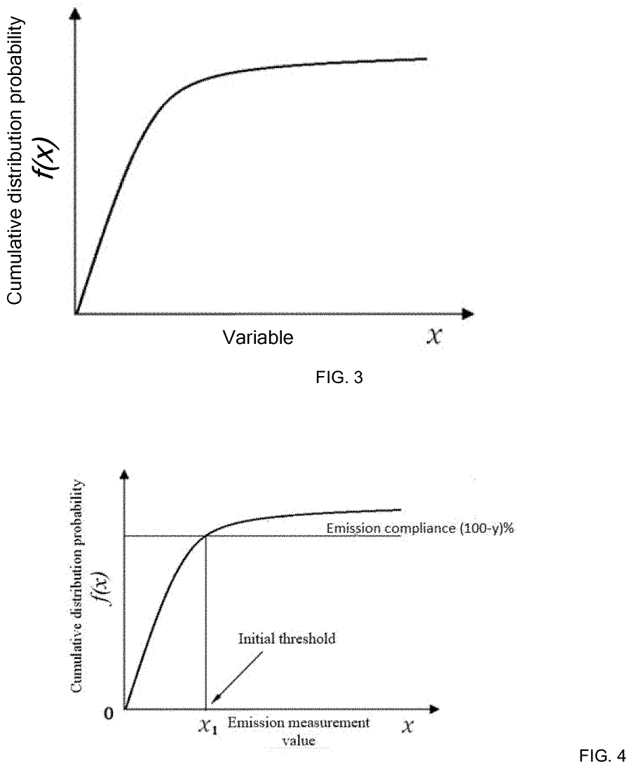

S101: Classify diesel vehicles into different types based on a volume of remote sensing test data of diesel vehicle emissions, as shown in FIG. 2. Diesel vehicles may be classified into light-duty diesel vehicles, medium-duty diesel vehicles and heavy-duty diesel vehicles according to their total mass, and may be further classified into light-duty diesel passenger vehicles, light-duty diesel trucks, medium-duty diesel passenger vehicles, medium-duty diesel trucks, heavy-duty diesel passenger vehicles and heavy-duty diesel trucks according to their uses.

S102: Divide a driving cycle of each type of diesel vehicles into i*j intervals using speed and acceleration as parameters, where the intervals are defined as Bin_{1,1}, Bin_{1,2} . . . Bin_{1,i}, Bin_{2,1} . . . B_{2,i} . . . Bin_{j-1,1} . . . Bin_{j-1,i} . . . Bin_{j,1} . . . Bin_{j,i}, respectively; each bin represents the interval-specific speed and acceleration of the vehicle under the driving cycle; i and j are set according to a specific situation.

S103: Detect the vehicle speed and acceleration through a vehicle driving state monitor of the vehicle emission remote sensing monitoring system, allocate the driving cycle parameters (vehicle speed and acceleration) and emission remote sensing test data to a corresponding bin, and perform statistical analysis on a vehicle emission remote sensing test result in each bin.

S104: Perform remote sensing detection of a NOₓ emission from a diesel vehicle. NO is dominant in the NOₓ emission of diesel engines, especially for diesel engines subject to the stage I, II and III emission standards of China, more than 90% of NOₓ emissions are NO. The detected NO₂ emission levels can be treated as NO equivalents. Diesel vehicles subject to China's stage IV emission standards are equipped with oxidation catalysts, which increases the NO₂ concentration in the NOₓ emissions, but the total NOₓ emissions can still be calculated by the method of the present disclosure.

A host computer calculates the NO emission of the diesel vehicle through data detected by an emission remote sensing instrument by using three methods, that is, by (1) detecting a concentration ratio of NO to CO₂, that is, NO/CO₂; (2) detecting a NO emission per unit mass of fuel (g/kg fuel); and (3) detecting an absolute concentration (ppm) of NO emission.

Method 1: Detect concentration ratios Q_{CO′} Q_{HC′} Q_{NO′} of CO, HC and NO to CO₂ in an exhaust plume respectively by the emission remote sensing instrument, and determine the combustion status of the vehicle engine and the NO emission level of the diesel vehicle by the concentration ratio Q_{NO} of NO to CO₂.

Method 2: Calculate the NO emission per unit mass of fuel (g/kg fuel) by the relative volume concentration ratios Q_{CO′} Q_{HC′} Q_{NO′} of CO, HC and NO to CO₂ and molecular weights of substances by

$${EF}_{NO} = \frac{30}{0.014} * \frac{{Q}_{NO}}{1 + {Q}_{CO} + 6 * {Q}_{HC}}$$

according to a carbon balance method, and determine the NO emission of the vehicle under inspection in real time, where,

$${Q}_{CO} = \frac{{C}_{CO}}{{C}_{{CO}^{2}}} , {Q}_{HC} = \frac{{C}_{{H}_{C}}}{{C}_{{CO}^{2}}} , {Q}_{NO} = \frac{{C}_{NO}}{{C}_{{CO}^{2}}}$$

Method 3: Calculate the concentrations of CO₂ and NO in the exhaust of the diesel vehicle by

$${EC}_{{CO}_{2}} = \frac{100}{0.5 {Q}_{HC} - 0.5 + AFR ( {Q}_{CO} + 4 {Q}_{HC} + 1 ) / 2.06}$$

$$and {EC}_{NO} = {EC}_{{CO}_{2}} * {Q}_{NO}$$

according to the concentration ratios Q_{CO′} Q_{HC′} Q_{NO′} of CO, HC and NO to CO₂ in the exhaust plume of the diesel vehicle and an AFR of the diesel engine in the test cycle, and determine the emissions of the vehicle under inspection in real time.

It can be seen that the remote sensing measurement of the NO emission of the diesel vehicle needs to obtain the relative volume concentration ratios of NO, CO and HC to CO₂ through the remote sensing test equipment, and to know the AFR of the diesel engine of the diesel vehicle under the test cycle. The AFR of the diesel engine of the diesel vehicle in a test cycle may be calculated by using two methods:

(1) A first method is to calculate cycle parameters of the diesel engine through driving cycle parameters of the vehicle, and determine the AFR of the diesel engine:

Calculate a tractive force of the diesel vehicle by

$${F}_{t} = {C}_{D} {A}_{f} \frac{{ρ}_{a}}{2} {( v \pm {v}_{w} )}^{2} + {mgC}_{R} cosφ + ma ( 1 + {ɛ}_{i} ) + mg sinφ ,$$

calculate an engine speed by

$$n = \frac{v \cdot {i}_{g} \cdot {i}_{0}}{0.337 \cdot r} ,$$

and calculate an engine torque by

$${T}_{t q} = \frac{{F}_{t} \cdot r}{{i}_{g} \cdot {i}_{0} \cdot {η}_{T}} ;$$

determine the AFR in the current engine cycle according to the engine torque, engine speed and an AFR map of the diesel engine.

In the equation, n represents an engine speed; V represents a vehicle speed; i_{g} represents a transmission gear ratio; i₀ represents a final drive ratio; r represents a rolling radius of a tire; η_{T} represents a mechanical efficiency of the transmission system; Fₜ represents a tractive force of the vehicle; C_{D} represents a drag coefficient; A_{f} represents a frontal area of the vehicle; ρₐ represents an air density; v represents a vehicle speed; v_{w} represents a wind speed; m represents a vehicle mass; a represents a vehicle acceleration; E represents a mass conversion coefficient of a rotating part of a powertrain; g represents an acceleration due to gravity; φ represents a road gradient; C_{R} represents a rolling resistance coefficient of a tire.

(2) A second method is to establish an AFR map model with diesel vehicle speed and acceleration as parameters based on the statistical analysis of diesel vehicle test data. The vehicle driving state monitor detects the speed and acceleration of the diesel vehicle. The host computer uses the speed v and acceleration a of the diesel vehicle as two-dimensional (2D) parameters, and obtains the AFR of the diesel vehicle in the current driving cycle through interpolation calculation.

S105: Perform remote sensing detection of an exhaust smoke opacity of a diesel vehicle. The remote sensing test of the exhaust smoke of the diesel vehicle uses ten pairs of optical paths provided by photoelectric sensors, where ten detection beams are emitted from one side, and received on the other side respectively. A light source works in a modulation mode, which effectively avoids the influence of ambient light on the measurement. The beam range covers the height of the tailpipe of most motor vehicles and can obtain a measurement result of a vertical section of the exhaust smoke of the diesel vehicle. The light transmittance is divided into 100 levels, and the remote sensing test result of the exhaust smoke is represented by opacity (%).

S106: Allocate the remote sensing test data of vehicle cycle parameters (vehicle speed and acceleration), NO emission and exhaust smoke opacity to corresponding bins by the vehicle remote sensing data monitoring platform; process the remote sensing test data of vehicle emissions by a probability distribution method for a discrete random variable in each bin of the vehicle cycle; take a cumulative probability value of a vehicle indicated as emission compliance by the cumulative distribution probability according to a proportion of high-emission diesel vehicles among the in-use diesel vehicles, and determine a corresponding emission measurement value as a determination threshold for screening high-emission vehicles.

S107: Perform statistical analysis of excess emission values of the diesel vehicle; record the diesel vehicle under inspection as one exceeding an emission limit if the remote sensing emission test result of the vehicle exceeds the high-emission determination threshold; if the vehicle's detection results of the same pollutant exceeds the high-emission determination threshold for a number of times that reach or exceed a specified number of times within a specified measurement period (for example, 6 months), determine the vehicle under inspection as a high-emission vehicle, where in this case, an owner of the vehicle may be notified to repair the vehicle; if no repair is made, a travel restriction may be imposed on the vehicle.

S108: Evaluate an average emission level of the vehicle by using a statistical average value of the emissions in each driving cycle bin of the diesel vehicle. The statistical average value of the big data of remote sensing test results has important practical significance. The statistical average value in each bin is sufficient to represent the true emission of this type of vehicle in the driving cycle bin and can be used to evaluate the vehicle emission level and estimate the emission.

S109: Delete the excess emission data of a diesel vehicle in the database after the vehicle passes an emission test after being repaired and record the excess emission information of the vehicle separately to evaluate the emission levels of various types of vehicles on the market.

S110: Perform statistical analysis regularly on the excess emission records of diesel vehicles in use by the vehicle remote sensing data monitoring platform, evaluate and rank the emission levels and emission control technology levels of all types of diesel vehicles on the market, and focus the spot check and supervision of emissions on a vehicle type with a high proportion of excess emission records.

S111: Since the big data processing system continues to screen out a certain proportion of high-emission vehicles and corresponding rectification measures are adopted, the average emissions of in-use vehicles are reduced. Over time, low-emission vehicles are increased, and old vehicles are eliminated, which constantly changes the composition of vehicle ownership in each emission stage. The big data of statistical analysis is updated in real time, and the mean and median values of the overall remote sensing emission data are updated simultaneously. This realizes the simultaneous update of the ownership composition of various in-use vehicles with the overall average emission level of vehicles and realizes the dynamic adjustment of the remote sensing determination threshold for screening high-emission vehicles. The high-emission determination threshold has the advantage of real-time updating compared with the annual emission inspection standard limits of in-use vehicles and is conducive to the scientific management of vehicle emission.

In overall, steps S101 to S111 involve the classification of diesel vehicles, the division of driving cycle bins of diesel vehicles, the remote sensing detection of NO emission of diesel vehicles, the remote sensing detection of the exhaust smoke of diesel vehicles, the statistical analysis of the remote sensing test big data of diesel vehicle emissions, the determination of high-emission diesel vehicles, the screening and supervision of high-emission vehicle types and the evaluation of the average emission level of diesel vehicles.

### Embodiment 3

The present disclosure provides a method for monitoring diesel vehicle emissions based on big data of remote sensing, which uses the system for monitoring diesel vehicle emissions based on big data of remote sensing provided by Embodiment 1. According to the different emission characteristics of diesel vehicles under different driving cycles, this monitoring method achieves the scientific and refined management of diesel vehicle emissions by classifying the diesel vehicles into different types and dividing a driving cycle into different bins. This embodiment is illustrated with reference to the emission remote sensing test of a certain type of diesel vehicles and the processing of the big data of emission remote sensing of the diesel vehicles in different driving cycle bins.

1) Diesel vehicles are classified into different types based on the volume of the remote sensing test data of diesel vehicle emissions and a need for refined management, as shown in FIG. 2. Diesel vehicles may be classified into light-duty diesel vehicles, medium-duty diesel vehicles and heavy-duty diesel vehicles according to their GVM and may be further classified into light-duty diesel passenger vehicles, light-duty diesel trucks, medium-duty diesel passenger vehicles, medium-duty diesel trucks, heavy-duty diesel passenger vehicles and heavy-duty diesel trucks according to their uses.

This embodiment is illustrated with reference to the emission remote sensing test result of a light-duty diesel passenger vehicle and the statistical analysis of the big data of the emission remote sensing of the light-duty diesel passenger vehicle in different driving cycle bins.

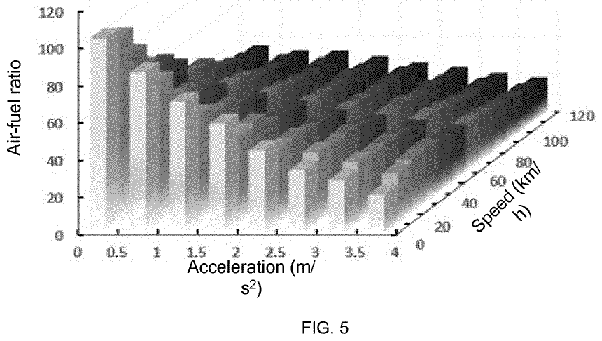

2) The driving cycle of the light-duty diesel passenger vehicle is divided into different bins, as shown in FIG. 5. When the diesel vehicle decelerates, the engine enters an idling mode or a forced idling mode. A fuel cut-off control strategy may be implemented, so that the exhaust smoke may be greatly reduced. Therefore, in this embodiment, the remote sensing test condition for the emission of the light-duty diesel passenger vehicle is set as a vehicle acceleration greater than or equal to zero. The speed in the bins of the driving cycle of this vehicle type is determined to be 0 to 120 km/h, and the acceleration is determined to be 0 to 4.0 m/s². In this embodiment, the driving cycle of this vehicle type is divided into 8*12 bins (Table 1). Statistical processing is performed on the AFR data of the light-duty diesel passenger vehicle under actual road conditions to obtain an average AFR in each bin of the driving cycle of the vehicle type, which is used for the subsequent inversion calculation of the remote sensing test of NOₓ emission. For the sake of convenience, the subsequent statistical analysis on the big data of emission remote sensing of the light-duty diesel passenger vehicle adopts the same bin method.

There are a total of 96 bins, including 12 speed bins, 10 km as a speed bin, and 8 acceleration bins, 0.5 m/s² as an acceleration bin. There is a statistical average value of AFR in each bin of the AFR map.

TABLE 1
Driving cycle bins of light-duty diesel passenger vehicle

| Bin No. | Speed (km/h) | Acceleration (m/s²) |
|---|---|---|
| Bin_{1, 1} | 0 ≤ v < 10 | 0 ≤ a < 0.5 |
| Bin_{1, 2} | 10 ≤ v < 20 | 0 ≤ a < 0.5 |
| Bin_{1, 3} | 20 ≤ v < 30 | 0 ≤ a < 0.5 |
| . . . | . . . | . . . |
| Bin_{1, 12} | 110 ≤ v ≤ 120 | 0 ≤ a < 0.5 |
| Bin_{2, 1} | 0 ≤ v < 10 | 0.5 ≤ a < 1 |
| Bin_{2, 2} | 10 ≤ v < 20 | 0.5 ≤ a < 1 |
| . . . | . . . | . . . |
| Bin_{2, 12} | 110 ≤ v ≤ 120 | 0.5 ≤ a < 1 |
| . . . | . . . | . . . |
| Bin_{8, 12} | 110 ≤ v ≤ 120 | 3.5 ≤ a ≤ 4 |

3) In this embodiment, a host computer takes images of the license plate and vehicle type by a license plate camera, identifies relevant information of the vehicle under inspection, including parameters such as vehicle type and vehicle configuration, and determines the type of the vehicle under inspection.

4) A vehicle driving state monitor detects the speed and acceleration of the diesel vehicle; the host computer uses the speed v and acceleration a of the diesel vehicle as 2D parameters and calculates the AFR of the diesel vehicle in the current driving cycle through interpolation.

5) The host computer calculates the NOₓ emission of the diesel vehicle by using three methods: (1) detect a concentration ratio of NO to CO₂, that is, NO/CO₂; (2) detect a NO emission per unit mass of fuel (g/kg fuel); and (3) detect an absolute concentration (ppm) of NO emission.

Method 1: Detect the emission concentration ratios of CO, HC and NOₓ to CO₂ in an exhaust plume, and determine the emission status of the vehicle under inspection in real time based on the emission concentration ratio of NOₓ to CO₂.

The relative volume concentration ratios of the gaseous emissions in the exhaust plume of the diesel vehicle are:

$$\begin{matrix}{Q}_{C0} = \frac{{C}_{CO}}{{C}_{{CO}^{2}}} & ( 1 ) \\ {Q}_{HC} = \frac{{C}_{{H}_{C}}}{{C}_{{CO}^{2}}} & ( 2 ) \\ {Q}_{C0} = \frac{{C}_{NO}}{{C}_{{CO}^{2}}} & ( 3 )\end{matrix}$$

where, Q_{NO}, Q_{CO} and Q_{HC} are respectively the relative volume concentration ratios of CO, HC and NO to CO₂ in the exhaust plume; C_{CO}, C_{HC}, C_{NO} and CO₂ are respectively the concentrations of CO, HC, NOₓ and CO₂ in the exhaust plume.

Method 2: Detect the NO emission per unit mass of fuel consumed by the diesel vehicle (g/kg fuel), and determine the emission status of the vehicle under inspection in real time.

According to a carbon balance method, the NO emission per unit mass of fuel consumed by the diesel vehicle is calculated as follows:

$${EF}_{NO} = \frac{30}{0.014} * \frac{{Q}_{NO}}{1 + {Q}_{CO} + 6 * {Q}_{HC}} ( g / kg fuel ) \qquad ( 4 )$$

Therefore, by detecting the NO emission per unit mass of fuel consumed by the diesel vehicle, the NO emission status of the vehicle under inspection can also be determined in real time.

Method 3: Detect an absolute concentration of NO emission in the exhaust plume of the diesel vehicle, and determine the emission status of the vehicle under inspection in real time.

The host computer calculates the emission concentration ratios of CO, HC and NOₓ to CO₂ in the exhaust plume of the diesel vehicle. The vehicle driving state monitor detects the speed and acceleration of the diesel vehicle. The host computer calculates the AFR of the diesel vehicle in the current driving cycle by performing interpolation calculation by using the speed and acceleration of the diesel vehicle as 2D parameters.

The volume percent concentration of CO₂ in the exhaust plume of the diesel vehicle is:

$${EC}_{{CO}_{2}} = \frac{100}{0.5 {Q}_{HC} - 0.5 + AFR ( {Q}_{CO} + 4 {Q}_{HC} + 1 ) / 2.06} \qquad ( 5 )$$

The absolute concentration (volume percent concentration) of NO in the exhaust plume of the diesel vehicle is calculated by:

EC_{NO}=EC_{CO}_{₂}*Q_{NO} (6)

Therefore, the real-time remote sensing measurement of gaseous emissions such as NO from the diesel vehicle needs to obtain the relative volume concentration ratios of NO, CO and HC to CO₂ through the remote sensing test equipment, and to know the AFR of the diesel engine during the combustion process in the test cycle.

The calculation method of the NO emission of the diesel vehicle is described with reference to Table 2. For a single remote sensing test of the NO emission of the diesel vehicle, the host computer performs 2D interpolation calculation by using the diesel vehicle's speed v and acceleration a as parameters to obtain the diesel vehicle's AFR in the current driving cycle. The host computer calculates the NO emission per unit mass of fuel (g/kg fuel) in the current driving cycle according to Eq. (4) and calculates the emission concentrations of CO₂ and NO in the exhaust plume of the diesel vehicle according to Eqs. (5) and (6). The calculation results are shown in Table 2.

TABLE 2
Calculation results of CO₂ and NO in the exhaust plume of diesel vehicle

| | | | | | | NO | | |
|---|---|---|---|---|---|---|---|---|
| | | | Concentration ratios by | | | emission | CO₂ | NO |
| Speed | Acceleration | AFR | remote sensing | | | factor | concentration | concentration |
| (km/h) | (m/s²) | calculated | Q_{CO} | Q_{HC} | Q_{NO} | (g/kg fuel) | (%) | (ppm) |
| 10 | 2.00 | 31.9 | 0.0144 | 0.0013 | 0.0227 | 31.89 | 6.37 | 1445.7 |
| 20 | 1.50 | 33.6 | 0.0156 | 0.0011 | 0.0174 | 33.61 | 6.04 | 1048.41 |
| 30 | 1.50 | 32.0 | 0.0145 | 0.001 | 0.0144 | 32.03 | 6.36 | 914.09 |
| 40 | 1.00 | 37.8 | 0.0152 | 0.0012 | 0.0164 | 37.75 | 5.36 | 878.15 |
| 50 | 0.05 | 41.3 | 0.0144 | 0.0013 | 0.0192 | 41.33 | 4.88 | 939.64 |
| 60 | 0.02 | 41.9 | 0.0138 | 0.001 | 0.0166 | 41.90 | 4.82 | 799.34 |
| 70 | 0.01 | 41.8 | 0.0131 | 0.0009 | 0.0167 | 41.76 | 4.84 | 807.62 |

The present disclosure proposes an inversion calculation method for the NO emission concentration in the exhaust plume of the diesel vehicle. The present disclosure calculates the NO emission concentration in the exhaust plume of the diesel vehicle through the pollutant information about the exhaust plume of the diesel vehicle measured by the emission remote sensing instrument and the AFR of the diesel engine in the test cycle. This method is suitable for the exhaust emission detection of various motor vehicles equipped with diesel engines, and features convenient, rapid and efficient operation.

6) The emission remote sensing instrument detects the exhaust smoke of the diesel vehicle.

The remote sensing test of the exhaust smoke of the diesel vehicle uses ten pairs of optical paths provided by photoelectric sensors, where ten detection beams are emitted from one side, and received on the other side respectively. A light source works in a modulation mode, which effectively avoids the influence of ambient light on the measurement. The beam range covers the height of the tailpipe of most motor vehicles and can obtain a measurement result of a vertical section of the exhaust smoke of the diesel vehicle. The light transmittance is divided into 100 levels, and the remote sensing test result of the exhaust smoke is represented by opacity (%).

7) The embodiment of the present disclosure provides a method for monitoring diesel vehicle emissions based on big data of remote sensing. The host computer sends the measured speed, acceleration, NOₓ emission, exhaust smoke opacity and vehicle type parameters of the diesel vehicle to the vehicle remote sensing data monitoring platform. The vehicle remote sensing data monitoring platform allocates the measured vehicle driving cycle parameters, NOₓ emission and exhaust smoke opacity to the driving cycle bins of this vehicle type based on the vehicle type, speed and acceleration parameters, for calculation and statistical analysis.

8) Statistical analysis is performed on the vehicle emission remote sensing test results in each bin of the driving cycle.

The exhaust smoke test of diesel vehicles under free acceleration is a traditional method of annual and random inspections of diesel vehicles in use. Since the free acceleration conditions are not normal driving conditions of diesel vehicles, a separate bin is set up, in which the exhaust smoke opacity of diesel vehicles under free acceleration is statistically analyzed.

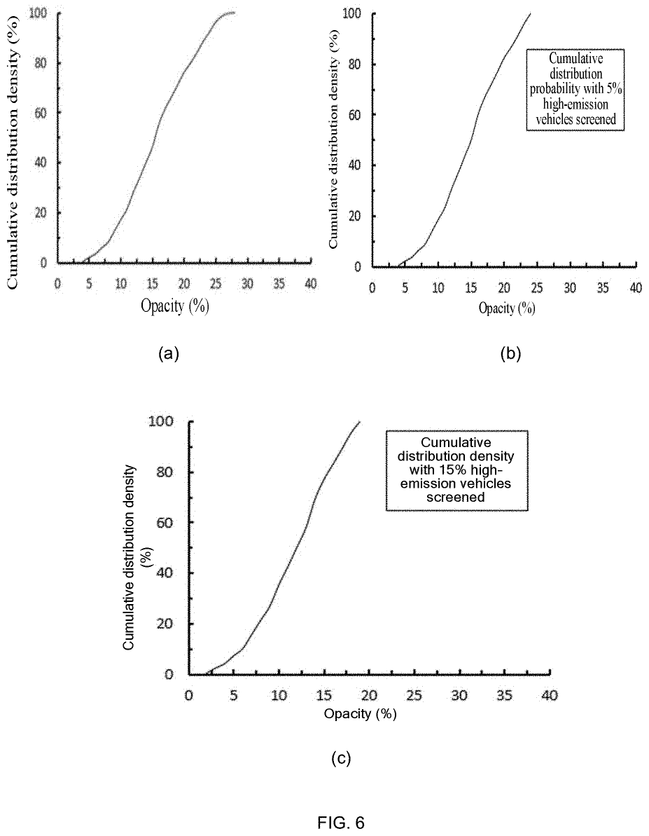

The detected opacity is treated as a discrete random variable, and the probability of each emission value is calculated to obtain the probability distribution density of each measured opacity. Then the cumulative distribution probability of the measured opacity is calculated. The cumulative distribution probability of the measured value of the exhaust smoke opacity of the light-duty diesel passenger vehicle under free acceleration is shown in FIG. 6.

9) Selection and Analysis of Emission Determination Threshold for High-Emission Diesel Vehicles

This embodiment is illustrated with reference to the statistical analysis result of the exhaust smoke opacity of the light-duty diesel passenger vehicle under free acceleration. According to a cumulative distribution probability curve of the remote sensing measurement values of exhaust smoke opacity of the light-duty diesel passenger vehicle under free acceleration, the screening threshold for the exhaust smoke opacity of high-emission vehicles under free acceleration is analyzed, as shown in Table 3.

TABLE 3
Analysis on the screening threshold for the exhaust smoke opacity
of high-emission diesel passenger vehicles under free acceleration

| Exhaust smoke opacity | | | Exhaust smoke opacity with | | Exhaust smoke opacity with | |
|---|---|---|---|---|---|---|
| under free | | | 5% high-emission vehicles | | 15% high-emission vehicles | |
| acceleration | | | screened | | screened | |
| | Maximum | Statistical | Maximum | Statistical | Maximum | Statistical |
| | value | average value | value | average value | value | average value |
| Smoke | 28 | 16.1 | 24 | 15.3 | 21 | 14.08 |
| opacity | | | | | | |

Due to the high rate of misjudgment in the current vehicle remote sensing test, the current remote sensing test only screens 5% of high-emission vehicles. In this embodiment, the analysis is carried out with the goal of individually controlling the exhaust smoke opacity under free acceleration.

Screening 5% high-emission vehicles means that, the emission measurement value with a cumulative distribution probability of 95% is taken from the cumulative probability distribution curve of the measurement value of the exhaust smoke opacity under free acceleration as the remote sensing determination threshold for screening high-emission diesel vehicles. If the remote sensing test value of the exhaust smoke opacity exceeds the screening threshold for high-emission vehicles in the bin, the inspected diesel vehicle is recorded as exceeding the emission limit.

The emission remote sensing test data and statistical analysis results in each bin are always in a dynamic update process, but for the convenience of analysis, they are handled as static temporarily. If the control target is to screen 5% high-emission vehicles, the determination threshold for the remote sensing test result of the exhaust smoke opacity of high-emission vehicles under free acceleration is 24%. When the determined high-emission vehicles meet the emission limit after maintenance, with the 5% high-emission vehicles screened, the exhaust smoke opacity under free acceleration is reduced from the maximum value of 28% to 24%, and the statistical average value of the exhaust smoke opacity is reduced from 16.1% to 15.3%.

In accordance with the determination principle of the emission limit in the revision standard of in-use vehicles, the proportion of high-emission vehicles is set between 10% and 20%. Taking 15% as an example, the emission measurement value corresponding to the cumulative distribution probability of 85% is taken, and the remote sensing determination threshold for screening the exhaust smoke opacity of high-emission vehicles is 21%. With the 15% high-emission vehicles screened, the exhaust smoke opacity under free acceleration is reduced from the maximum value of 28% to 21%, and the statistical average value of the exhaust smoke opacity is reduced from 16.1% to 14.08%.

10) Comprehensive Analysis of Emission Determination Threshold for High-Emission Diesel Vehicles

The emission remote sensing test value of each diesel vehicle is composed of a set of data (NO emission and exhaust smoke opacity). There is a situation in which one of the two parameters exceeds the emission limit while the other meets the emission limit or both of them exceed the emission limit. Therefore, if the NO emission and smoke opacity of a vehicle are screened according to the 5% high-emission vehicle determination threshold, and when one of them exceeds the emission limit, the vehicle is determined as a high-emission vehicle, the actual proportion of high-emission vehicles screened is greater than 5%. If a vehicle is determined as a high-emission vehicle only when both parameters exceed the emission limit, the actual proportion of high-emission vehicles screened is less than 5%. Therefore, it is necessary to appropriately adjust the determination thresholds of NO emission and exhaust smoke opacity for high-emission vehicles through subsequent tests, so that the proportion of high-emission vehicles actually screened is controlled to 5%.

11) Determination of High-Emission Diesel Vehicles

If the number of excess emission records reaches a determination number within a specified time period, for example, referring to the HJ 845-2017 standard, the remote sensing test values of the same pollutant exceed the emission limit specified by the standard twice or more and the measurement interval is within 6 natural months, the vehicle under inspection is determined unqualified and as a high-emission vehicle. The owner of the vehicle under inspection may be notified to repair the vehicle. If no repair is made, a travel restriction may be imposed.

When the vehicle passes the emission test after being repaired, the excess emission data of the vehicle is deleted from the database and recorded separately for comprehensive evaluation of the emission levels of various types of vehicles.

12) Comprehensive Evaluation of Emission Levels of Various Types of Vehicles in Use

The vehicle remote sensing data monitoring platform regularly counts the excess emission records of various types of vehicles and ranks and evaluates the number of excess emissions records and emission levels of all types of vehicles on the market. The spot check and supervision of emissions focuses on a vehicle type with a high proportion of excess emission records.

13) The statistical average value of each bin is used to evaluate the vehicle emission level.

The statistical average value of the remote sensing monitoring data in the bin of the vehicle driving cycle may be calculated by a discrete random variable equation:

$$\overline{x} = {\sum }_{i = 1}^{n} {x}_{i} {p}_{i} \qquad ( 7 )$$

The statistical average of remote sensing monitoring data is the weighted average of all data, which considers not only the value of each remote sensing monitoring data, but also the probability of the value.

With the rapid increase in the volume of the remote sensing test data stored in each bin, the statistical average value obtained by using the probability statistical analysis method represents the true emission value of this type of vehicle in the driving cycle bin, which can be used for vehicle emission level evaluation and emission estimation. At this time, the fluctuation of the random emission test result around the statistical average has a negligible impact on the statistical average of each bin, and the statistical average of each bin has excellent robustness.

14) Dynamic Update of Remote Sensing Emission Determination Threshold in Each Bin

The remote sensing emission data in each driving cycle bin of the vehicle is updated in real time. The screening of high-emission vehicles is carried out repeatedly, and the detected high-emission vehicles are subject to mandatory maintenance or elimination, which reduces the overall average emissions of the vehicles. Over time, as low-emission vehicles are increased and old vehicles are eliminated, the composition of vehicle ownership is changing. Due to the real-time update of the statistical analysis of the remote sensing test data, the remote sensing emission determination threshold for screening high-emission vehicles is dynamically adjusted, changing simultaneously with the average emission levels of vehicles and the composition of vehicle ownership. The high-emission determination threshold has the advantage of real-time updating compared with the annual emission inspection standard limits of in-use vehicles.

### Embodiment 4

The present disclosure provides a system for monitoring diesel vehicle emissions based on big data of remote sensing. The monitoring system includes: a vehicle remote sensing data monitoring platform, a host computer, an emission remote sensing instrument, a vehicle driving state monitor, an information display screen and a license plate camera. The emission remote sensing instrument, the vehicle driving state monitor, the information display screen and the license plate camera are all connected to the host computer; the host computer is connected to the vehicle remote sensing data monitoring platform via the Internet.

The emission remote sensing instrument is used to acquire information of a pollutant in an exhaust plume of a vehicle under inspection.

The vehicle driving state monitor is used to acquire a speed and an acceleration of the vehicle under inspection.

The information display screen is used to display relevant information of the vehicle under inspection.

The license plate camera is used to capture license plate information of the vehicle under inspection.

The host computer is used to calculate a concentration of the pollutant in the exhaust plume of the vehicle under inspection based on the information of the pollutant in the exhaust plume, and pre-store information of all diesel vehicles, different driving cycle bins of each type of diesel vehicles and high-emission thresholds set for different bins.

The vehicle remote sensing data monitoring platform is used to determine a corresponding bin for the vehicle under inspection based on the speed and acceleration of the vehicle under inspection and determine whether the vehicle under inspection is a high-emission vehicle based on the concentration of the pollutant in the exhaust plume and the high-emission threshold set in the corresponding bin of the vehicle under inspection.

### Embodiment 5

The present disclosure provides a method for monitoring diesel vehicle emissions based on big data of remote sensing. The monitoring method includes:

Acquire a speed and an acceleration of a vehicle under inspection by a vehicle driving state monitor.

Capture license plate information of the vehicle under inspection by a license plate camera.

Acquire pollutant data about an exhaust plume of the vehicle under inspection, where the pollutant data includes a concentration ratio of CO to CO₂, a concentration ratio of HC to CO₂ and a concentration ratio of NO to CO₂.

Determine an AFR of a diesel engine of the vehicle under inspection based on the speed and acceleration of the vehicle under inspection and the pollutant data about the exhaust plume.

Determine a NO emission level according to the AFR.

Calculate an exhaust smoke opacity of the vehicle under inspection according to an opacity of the exhaust plume.

Determine a bin for the vehicle under inspection according to the type, speed, acceleration, NO emission level and exhaust smoke opacity of the vehicle under inspection.

Determine whether the vehicle under inspection is a high-emission vehicle according to the NO emission level, the exhaust smoke opacity and a high-emission threshold in the bin of the vehicle under inspection.

Each embodiment in the specification of the present disclosure is described in a progressive manner. Each embodiment focuses on the difference from other embodiments, and the same and similar parts between the embodiments may refer to each other. For a device disclosed in the embodiments, since it corresponds to the method disclosed in the embodiments, the description is simple, and reference can be made to the method description.

The above illustration of the disclosed embodiments makes it possible for a person skilled in the art to implement or practice the present disclosure. Various modifications to these embodiments are readily apparent to a person skilled in the art, and the generic principles defined herein may be practiced in other embodiments without departing from the spirit or scope of the disclosure. Thus, the present disclosure is not limited to the embodiments shown herein but falls within the widest scope consistent with the principles and novel features disclosed herein.

What is claimed is:

1. A system for monitoring diesel vehicle emissions based on big data of remote sensing, comprising: a vehicle remote sensing data monitoring platform, a host computer, an emission remote sensing instrument, a vehicle driving state monitor, an information display screen and a license plate camera, wherein the emission remote sensing instrument, the vehicle driving state monitor, the information display screen and the license plate camera are all connected to the host computer; the host computer is connected to the vehicle remote sensing data monitoring platform via the Internet;
the emission remote sensing instrument is used to acquire information of a pollutant in an exhaust plume of a vehicle under inspection;
the vehicle driving state monitor is used to acquire a speed and an acceleration of the vehicle under inspection;
the information display screen is used to display relevant information of the vehicle under inspection;
the license plate camera is used to capture license plate information of the vehicle under inspection;
the host computer is used to acquire information, process data and calculate a concentration of the pollutant in the exhaust plume;
the vehicle remote sensing data monitoring platform is used to receive vehicle cycle information and emission data transmitted by the host computer, determine a high-emission vehicle and store relevant information to a database, wherein the database pre-stores information of all diesel vehicles, different driving cycle bins of each type of diesel vehicles and high-emission thresholds set for different bins.

2. The system for monitoring diesel vehicle emissions based on big data of remote sensing according to claim 1, wherein the emission remote sensing instrument adopts a vertical or horizontal optical path, and is disposed in a passing area of a vehicle; the emission remote sensing instrument comprises a detection light emitting device, a detection light receiving device and a detection light reflecting device; the detection light emitting device and the detection light reflecting device are disposed oppositely; the detection light emitting device and the detection light receiving device are located on the same side; the detection light emitting device and the detection light receiving device are both connected with the host computer;
the detection light emitting device is used to emit detection light;
the detection light reflecting device is used to reflect the detection light to the detection light receiving device;
the detection light receiving device is used to detect an intensity of the detection light passing through the exhaust plume.

3. A method for monitoring diesel vehicle emissions based on big data of remote sensing, comprising:
step 1: detecting a speed and an acceleration of a vehicle under inspection through a vehicle driving state monitor, detecting an intensity of detection light passing through an exhaust plume by an emission remote sensing instrument, capturing license plate information of the vehicle under inspection by a license plate camera, and transmitting the above information to a host computer;
step 2: calculating concentration ratios of CO, HC and NO to CO₂ in the exhaust plume by the host computer according to the detection light intensity detected by the emission remote sensing instrument; determining an air-fuel ratio (AFR) of a diesel engine of the vehicle under inspection according to the speed, acceleration and type of the vehicle under inspection, determining a NO emission level, and calculating an exhaust smoke opacity of the diesel vehicle based on an opacity of the exhaust plume; and
step 3: receiving, by a vehicle remote sensing data monitoring platform, the type, driving cycle and emission information of the vehicle under inspection sent by the host computer; determining the type of the vehicle under inspection; allocating the vehicle's driving cycle parameters, NO emission and exhaust smoke opacity data to a bin corresponding to the vehicle type according to the vehicle speed and acceleration; performing statistical analysis on the emission data, and screening a high-emission vehicle, to realize supervision of the high-emission vehicle.

4. The method for monitoring diesel vehicle emissions based on big data of remote sensing according to claim 3, wherein in step 2, the determining a NO emission level comprises determining a concentration ratio of NO to CO₂ or determining a NO emission per unit mass of fuel consumed or determining an absolute concentration of NO in the exhaust plume.

5. The method for monitoring diesel vehicle emissions based on big data of remote sensing according to claim 4, wherein the concentration ratio of NO to CO₂ is Q_{NO}:
$${Q}_{NO} = \frac{{C}_{NO}}{{C}_{{CO}_{2}}} \qquad ( 1 )$$
wherein, C_{NO} and C_{CO}_{₂} represent concentrations of NO and CO2 in the exhaust plume, respectively.

6. The method for monitoring diesel vehicle emissions based on big data of remote sensing according to claim 4, wherein the NO emission per unit mass of fuel consumed is EF_{NO}:
$${EF}_{NO} = \frac{30}{0.014} * \frac{{Q}_{NO}}{1 + {Q}_{CO} + 6 * {Q}_{HC}} \qquad ( 2 )$$
wherein, Q_{NO}, Q_{CO} and Q_{HC} represent the concentration ratios of NO to CO₂, CO to CO₂ and HC to CO₂ in the exhaust plume, respectively;
$$\begin{matrix}{Q}_{CO} = \frac{{C}_{CO}}{{C}_{{CO}_{2}}} & ( 3 ) \\ {Q}_{{H}_{C}} = \frac{{C}_{{H}_{C}}}{{C}_{{CO}_{2}}} & ( 4 ) \\ {Q}_{NO} = \frac{{C}_{NO}}{{C}_{{CO}_{2}}} & ( 5 )\end{matrix}$$
wherein, C_{NO}, C_{CO}_{₂}, C_{CO} and C_{HC} represent the concentrations of NO, CO₂, CO and HC in the exhaust plume, respectively.

7. The method for monitoring diesel vehicle emissions based on big data of remote sensing according to claim 4, wherein the determining an absolute concentration of NO in the exhaust plume specifically comprises:
step a: calculating a volume percent concentration of CO₂ in the exhaust plume:
$${EC}_{{CO}_{2}} = \frac{100}{0.5 {Q}_{HC} - 0.5 + AFR ( {Q}_{CO} + 4 {Q}_{HC} + 1 ) / 2.06} \qquad ( 6 )$$
wherein, AFR represents an air-fuel ratio; Q_{CO} and Q_{HC} respectively represent concentration ratios of CO to CO₂ and HC to CO₂ in the exhaust plume,
$${Q}_{CO} = \frac{{C}_{CO}}{{C}_{{CO}_{2}}} , {Q}_{{H}_{C}} = \frac{{C}_{{H}_{C}}}{{C}_{{CO}_{2}}} ;$$
step b: calculating the absolute concentration of NO in the exhaust plume:
EC_{NO}=EC_{CO}_{₂}*Q_{NO} (7)
wherein, Q_{NO} represents a concentration ratio of NO to CO₂,
$${Q}_{NO} = \frac{{C}_{NO}}{{C}_{{CO}_{2}}} .$$

8. The method for monitoring diesel vehicle emissions based on big data of remote sensing according to claim 7, wherein the AFR is calculated by using two methods, among which a first calculation method comprises:
step a: converting the vehicle speed and acceleration into an engine speed and an engine torque respectively through a vehicle dynamics model;
$$n = \frac{v \cdot {i}_{g} \cdot {i}_{0}}{0.337 \cdot r} \qquad ( 8 )$$
wherein, n represents an engine speed; v represents a vehicle speed; i_{g} represents a transmission gear ratio; i₀ represents a final drive ratio; r represents a rolling radius of a tire;
$${T}_{tq} = \frac{{F}_{t} \cdot r}{{i}_{g} \cdot {i}_{0} \cdot {η}_{T}} \qquad ( 9 )$$
wherein, η_{T} represents a mechanical efficiency of the transmission system, and Fₜ represents a tractive force of the vehicle; the tractive force of the vehicle is calculated according to a vehicle dynamics equation:
$${F}_{t} = {C}_{D} {A}_{f} \frac{{ρ}_{a}}{2} {( v \pm {v}_{w} )}^{2} + {mgC}_{R} cos φ + ma ( 1 + {ɛ}_{i} ) + m g sin φ \qquad ( 10 )$$
wherein, C_{D} represents a drag coefficient; A_{f} represents a frontal area of the vehicle; ρₐ represents an air density; v represents a vehicle speed; v_{w} represents a wind speed; m represents a vehicle mass; a represents a vehicle acceleration; εᵢ represents a mass conversion coefficient of a rotating part of a powertrain; g represents an acceleration due to gravity; φ represents a road gradient; C_{R} represents a rolling resistance coefficient of a tire;
step b: establishing an AFR map with the engine torque and speed as parameter variables, and interpolating the engine torque and speed of the diesel vehicle under the test cycle to the AFR map to obtain an instantaneous AFR of the engine of the diesel vehicle under the test cycle;
a second calculation method comprises:
substituting the speed and acceleration of the vehicle under inspection as two-dimensional (2D) parameters into a pre-stored AFR map model established with speed and acceleration as parameters, and performing interpolation calculation to obtain the AFR of the diesel vehicle in the current driving cycle.

9. The method for monitoring diesel vehicle emissions based on big data of remote sensing according to claim 5, wherein the different driving cycle bins of each type of diesel vehicles and the high-emission thresholds set for different bins are determined as follows:
step a: classifying diesel vehicles according to a gross vehicle mass (GVM);
step b: dividing a driving cycle of each type of diesel vehicles into i*j intervals using speed and acceleration as parameters according to a volume of remote sensing test data and a need for monitoring, each interval being a bin;
step c: processing emission data by using a probability distribution method for a discrete random variable:
taking the emission remote sensing test data of the vehicle under inspection as a discrete random variable, letting x₁, x₂, . . . , xₙ be values of the emission data discrete variable x and p₁, p₂, . . . , pₙ be probabilities corresponding to these values, calculating probability distribution of discrete remote sensing test data xᵢ in real time, wherein the probability distribution of remote sensing test data xᵢ is expressed as:
P(x₁)=pᵢ (11)
wherein, i=1, 2, . . . , n;
probability pᵢ satisfies
$${\sum }_{i = 1}^{n} {p}_{i} = 1 \qquad ( 12 )$$
deriving a cumulative distribution probability of the discrete emission data variable x by a probability distribution function f(x):
f(xᵢ)=Σ₁ⁱpᵢ (13)
calculating a probability of a value of the discrete emission data variable x that falls within [a,b] by:
P(a≤x<b)=f(b)−f(a) (14)
setting a proportion of high-emission vehicles as y %, and taking an emission measurement value with a cumulative distribution probability of (100−y) % as an emission determination threshold for screening a high-emission vehicle.

10. The method for monitoring diesel vehicle emissions based on big data of remote sensing according to claim 6, wherein the different driving cycle bins of each type of diesel vehicles and the high-emission thresholds set for different bins are determined as follows:
step a: classifying diesel vehicles according to a gross vehicle mass (GVM);
step b: dividing a driving cycle of each type of diesel vehicles into i*j intervals using speed and acceleration as parameters according to a volume of remote sensing test data and a need for monitoring, each interval being a bin;
step c: processing emission data by using a probability distribution method for a discrete random variable:
taking the emission remote sensing test data of the vehicle under inspection as a discrete random variable, letting x₁, x₂, . . . xₙ be values of the emission data discrete variable x and p₁, p₂, . . . pₙ be probabilities corresponding to these values, calculating probability distribution pᵢ of discrete remote sensing test data xᵢ in real time, wherein the probability distribution of remote sensing test data xᵢ is expressed as:
P(xᵢ)=pi (11)
wherein, i=1, 2, . . . , n;
probability pᵢ satisfies
$${\sum }_{i = 1}^{n} {p}_{i} = 1 \qquad ( 12 )$$
deriving a cumulative distribution probability of the discrete emission data variable x by a probability distribution function f(x):
f(xᵢ)Σ₁ⁱpᵢ (13)
calculating a probability of a value of the discrete emission data variable x that falls within [a,b] by:
P(a≤x<b)=f(b)−f(a) (14)
setting a proportion of high-emission vehicles as y %, and taking an emission measurement value with a cumulative distribution probability of (100−y) % as an emission determination threshold for screening a high-emission vehicle.

11. The method for monitoring diesel vehicle emissions based on big data of remote sensing according to claim 7, wherein the different driving cycle bins of each type of diesel vehicles and the high-emission thresholds set for different bins are determined as follows:
step a: classifying diesel vehicles according to a gross vehicle mass (GVM);
step b: dividing a driving cycle of each type of diesel vehicles into i*j intervals using speed and acceleration as parameters according to a volume of remote sensing test data and a need for monitoring, each interval being a bin;
step c: processing emission data by using a probability distribution method for a discrete random variable:
taking the emission remote sensing test data of the vehicle under inspection as a discrete random variable, letting x₁, x₂, . . . xₙ be values of the emission data discrete variable x and p₁, p₂, . . . pₙ be probabilities corresponding to these values, calculating probability distribution pᵢ of discrete remote sensing test data xᵢ in real time, wherein the probability distribution of remote sensing test data xᵢ is expressed as:
P(xᵢ)=pᵢ (11)
wherein, i=1, 2, . . . , n;
probability pi satisfies
$${\sum }_{i = 1}^{n} {p}_{i} = 1 \qquad ( 12 )$$
deriving a cumulative distribution probability of the discrete emission data variable x by a probability distribution function f(x):
f(xᵢ)=Σ₁ⁱpᵢ (13)
calculating a probability of a value of the discrete emission data variable x that falls within [a,b] by:
P(a≤x<b)=f(b)−f(a) (14)
setting a proportion of high-emission vehicles as y %, and taking an emission measurement value with a cumulative distribution probability of (100−y) % as an emission determination threshold for screening a high-emission vehicle.

12. The method for monitoring diesel vehicle emissions based on big data of remote sensing according to claim 8, wherein the different driving cycle bins of each type of diesel vehicles and the high-emission thresholds set for different bins are determined as follows:
step a: classifying diesel vehicles according to a gross vehicle mass (GVM);
step b: dividing a driving cycle of each type of diesel vehicles into i*j intervals using speed and acceleration as parameters according to a volume of remote sensing test data and a need for monitoring, each interval being a bin;
step c: processing emission data by using a probability distribution method for a discrete random variable:
taking the emission remote sensing test data of the vehicle under inspection as a discrete random variable, letting x₁, x₂, . . . xₙ be values of the emission data discrete variable x and p₁, p₂, . . . pₙ be probabilities corresponding to these values, calculating probability distribution pᵢ of discrete remote sensing test data xᵢ in real time, wherein the probability distribution of remote sensing test data xᵢ is expressed as:
P(xᵢ)=pᵢ
wherein, i=1, 2, . . . , 3;
probability pᵢ satisfies
$${\sum }_{i = 1}^{n} {p}_{i} = 1 \qquad ( 12 )$$
deriving a cumulative distribution probability of the discrete emission data variable x by a probability distribution function f(x):
f(xᵢ)Σ₁ⁱpᵢ (13)
calculating a probability of a value of the discrete emission data variable x that falls within [a,b] by:
P(a≤x<b)=f(b)−f(a)
setting a proportion of high-emission vehicles as y %, and taking an emission measurement value with a cumulative distribution probability of (100−y) % as an emission determination threshold for screening a high-emission vehicle.

13. The method for monitoring diesel vehicle emissions based on big data of remote sensing according to claim 9, wherein if the NO emission level or exhaust smoke opacity of a vehicle under inspection exceeds the high-emission threshold set in the driving cycle bin corresponding to the vehicle type, it indicates that the emission of the vehicle under inspection exceeds an emission standard.

14. The method for monitoring diesel vehicle emissions based on big data of remote sensing according to claim 10, wherein if the NO emission level or exhaust smoke opacity of a vehicle under inspection exceeds the high-emission threshold set in the driving cycle bin corresponding to the vehicle type, it indicates that the emission of the vehicle under inspection exceeds an emission standard.

15. The method for monitoring diesel vehicle emissions based on big data of remote sensing according to claim 11, wherein if the NO emission level or exhaust smoke opacity of a vehicle under inspection exceeds the high-emission threshold set in the driving cycle bin corresponding to the vehicle type, it indicates that the emission of the vehicle under inspection exceeds an emission standard.

16. The method for monitoring diesel vehicle emissions based on big data of remote sensing according to claim 12, wherein if the NO emission level or exhaust smoke opacity of a vehicle under inspection exceeds the high-emission threshold set in the driving cycle bin corresponding to the vehicle type, it indicates that the emission of the vehicle under inspection exceeds an emission standard.

17. A system for monitoring diesel vehicle emissions based on big data of remote sensing, comprising: a vehicle remote sensing data monitoring platform, a host computer, an emission remote sensing instrument, a vehicle driving state monitor, an information display screen and a license plate camera, wherein the emission remote sensing instrument, the vehicle driving state monitor, the information display screen and the license plate camera are all connected to the host computer; the host computer is connected to the vehicle remote sensing data monitoring platform via the Internet;
the emission remote sensing instrument is used to acquire information of a pollutant in an exhaust plume of a vehicle under inspection;
the vehicle driving state monitor is used to acquire a speed and an acceleration of the vehicle under inspection;
the information display screen is used to display relevant information of the vehicle under inspection;
the license plate camera is used to capture license plate information of the vehicle under inspection;
the host computer is used to calculate a concentration of the pollutant in the exhaust plume of the vehicle under inspection based on the information of the pollutant in the exhaust plume, and pre-store information of all diesel vehicles, different driving cycle bins of each type of diesel vehicles and high-emission thresholds set for different bins;
the vehicle remote sensing data monitoring platform is used to determine a corresponding bin for the vehicle under inspection based on the speed and acceleration of the vehicle under inspection, and determine whether the vehicle under inspection is a high-emission vehicle based on the concentration of the pollutant in the exhaust plume and the high-emission threshold set in the corresponding bin of the vehicle under inspection.
